(19) United States
(12) Patent Application Publication
(10) Pub. No.: US 2020/0155028 A1
(43) Pub. Date: May 21, 2020

(54) System and Method for Characterizing Arrhythmias

(71) Applicant: Rutgers, The State University of New Jersey, New Brunswick, NJ (US)

(72) Inventor: Amardeep Saluja, Brooklyn, NY (US)

(21) Appl. No.: 16/092,282

(22) PCT Filed: Apr. 19, 2017
(86) PCT No.: PCT/US2017/028274

§ 371 Date: Oct. 9, 2018

Related U.S. Application Data
(60) Provisional application No. 62/324,510, filed on Apr. 19, 2016.

(51) Int. Cl. A61B 5/0464; A61B 5/042; A61B 5/04

(52) U.S. Cl. CPC A61B 5/0464; A61B 5/04011; A61B 5/0422

(57) ABSTRACT

A system and method of discovering the origin of a wave front in a human heart by measuring the difference in time of the activation of electrodes due to the propagation of the wave front through the heart. The location of the origin can then be mathematically modelled using the knowledge of the distance between the electrodes and the difference in time of activation.

### RELATED APPLICATIONS

This application claims the benefit of U.S. Provisional Patent Application No. 62/324,510, filed Apr. 19, 2016.

### BACKGROUND OF THE INVENTION

Cardiac arrhythmia is a condition in which the heartbeat is irregular, too fast, or too slow. Tachycardia is a heart rate that is too fast, usually above 100 beats per minute in adults, and bradycardia is a heart rate that is too slow, usually below 60 beats per minute in adults.

There are two types of wave fronts that can cause tachycardia. One is a focal, in which the focus of the tachycardia is a part of the heart that is beating abnormally, for whatever reason, causing the wave front to radiate outwardly in all directions from a focus. The second type is a re-entrant tachycardia, in which an electrical impulse enters a “circuit” and travels around the circuit. One can consider the focus of the tachycardia (i.e., the origin of the wave front) in this case is the point where the electrical impulse exits the circuit.

With respect to tachycardia, it is desirable, to provide treatment, to find the origin of the wave front that is causing the tachycardia, regardless of whether the tachycardia is focal or re-entrant. One traditional method of wave front localization includes entrainment (or overdrive pacing when applied to a focal tachycardia, henceforth also referred to also as entrainment). In traditional entrainment, the interval between the last paced beat and the first return signal as recorded in the pacing catheter (the PPI) approaches the tachycardia cycle length (TCL) as the site of pacing approaches the tachycardia circuit.

While useful, this approach is limited in that it is only is capable of analyzing one point at a time, and considers only the information available in the pacing catheter. Additional data can only be obtained with successive entrainment maneuvers, which may be time consuming and which may result in termination or transformation of the tachycardia circuit.

Another method of localizing arrhythmia wave fronts involves mapping local activation, typically by using three dimensional elecroanatomic software. While also useful, this process can be time-consuming and depends on the arrhythmia persisting long enough to provide a complete map.

### SUMMARY OF THE INVENTION

By describing the relationship between the distance between bipole pairs, the timing of the response in non-pacing electrodes remote to entrainment, and the relative activation of recoding bipoles during tachycardia, it is possible to rapidly discover the source of the wave front, and thus the origin of the tachycardia. More generally, the method will work in localizing any type of wave front propagating through the heart.

The system and method of the invention requires the insertion of multiple electrodes into the heart and a system capable of reading waveforms from the electrodes. In one embodiment of the invention, it is also necessary to pace from one of the pair of electrodes until entrainment is achieved. This is used to discover the distance between pairs of electrodes.

Two methods to obtain information about tachycardias are used.

Method A. Bipoles distal to the pacing site can be used to estimate the proximity of the recording site to the tachycardia circuit, provided those bipoles are recording antidromic activity.

Conventionally, the interval between the last paced beat and the first return electrogram (EGM) is called the post-pacing interval (PPI). The term derived PPI, or dPPI, is the interval between the last entrained EGM in an unpaced electrode pair and the first return cycle length, as shown in FIG. 1.

The relationship between the timings observed in the pacing and recording electrodes and their interaction with other characteristics of the tachycardia can be described mathematically. This mathematical model can then be used to predict the proximity of the recording electrode to the tachycardia origin.

Method B. The relationship between the distance between two bipoles and their activation during tachycardia can be mathematically described. When the pattern of linear activation is known (such as is determined during antidromic activation during entrainment or sinus rhythm pacing), this information can be used to estimate the distance between the bipoles, and the mathematical description allows for the prediction of tachycardia origin.

Computer software systems allow operators to track the location of catheters in space in the heart, as well as to automatically mark electrogram locations. By keeping track of the information generated by this method and the locations of the catheters at the time the information is collected, tachycardias can be rapidly characterized in three-dimensional space.

In another embodiment of the invention, pacing is unnecessary because the distance between electrodes can be determined by other, software-assisted means.

In all embodiments of the invention, once the distance between two given pairs of electrodes and the difference in activation time between that pair of electrodes is known, the mathematical relationship can be plotted on a three dimensional electroanatomical model of the heart. Once this is done with several pairs of electrodes, the intersection of the plots will reveal the origin of the wave front.

### DESCRIPTION OF THE DRAWINGS

FIG. 1 is a graph showing entrainment pacing.

FIG. 2 is a graph showing the antidromic activation of the recording site during entrainment.

FIG. 3 is a graph showing the measurement of the timing of linear activation versus tachycardia activation.

FIG. 4 is a schematic diagram showing antidromic activation during entrainment, and the various components involved.

FIG. 5 is an idealized model showing the relationship between the distance between bipoles B and A and the difference in activation timing during tachycardia.

FIGS. 6(A, B) show the application of a first embodiment of the invention (Method A), as described below.

FIGS. 7(A, B, C) show the application of a second embodiment of the invention (Method B), as described below.

### DETAILED DESCRIPTION

In a situation where the distance between bipoles must be determined experimentally, the following methods can be used.

An entrainment maneuver is performed. When the conventional PPI at the pacing site shows it to be outside of the circuit, antidromically activated sites are assessed among the available unpaced bipoles. Antidromically activated sites are identified by measuring the last entrained and the first return electrograms (EGMs) in each channel.

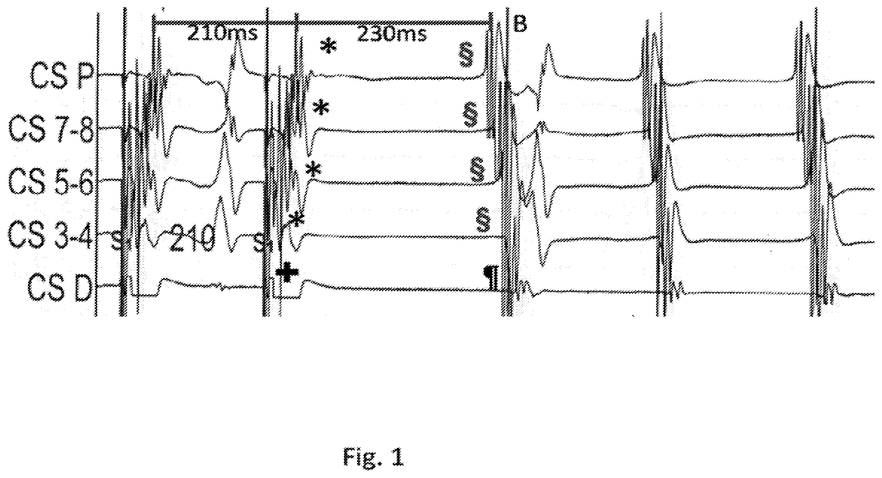

The last entrained EGM is the EGM that terminates an interval approximately equal to the paced cycle length. The following EGM is the first return EGM in that channel. As shown in FIG. 1, entrainment pacing is done from the CS D channel during an arrhythmia. The EGMs marked with an “*” terminate intervals that equal the pacing cycle length (210 ms), and represent the last entrained EGMs. The EGMs that follow in each channel, marked with the symbol “§” are the first return EGMs in that channel. The conventional PPI at CS D is >TCL.

The last entrained EGMs in each of the recording channels (CS 34, CS 56, CS 78, CS P) come after activation at the site of pacing (i.e. the order of EGMs is [symbol] , *). During native tachycardia, the EGMs in each of those channels come before the EGM in CS D (i.e, the order of EGMs is §, ¶). This change in activation orientation between entrainment and native tachycardia defines antidromic activation of the recording sites.

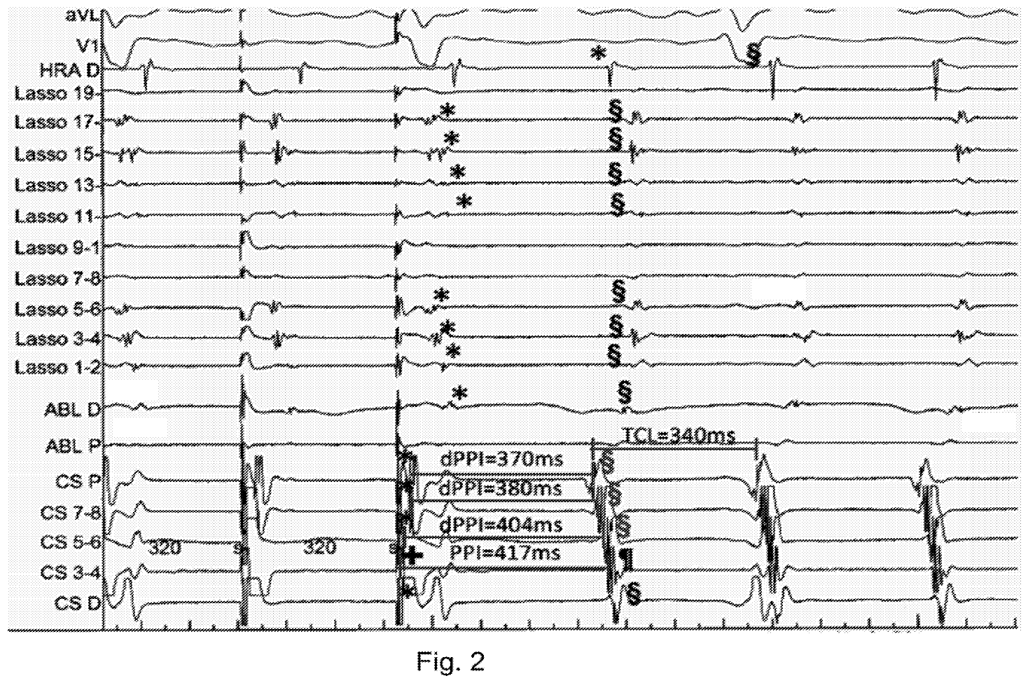

To discover antidromically activated EGMs relative to the site of pacing, determine if the sequence of activation of EGMs on recorded bipoles relative to the paced bipole is opposite for the last entrained beat versus native tachycardia. If so, the recording site is antidromically activated during entrainment, as shown in FIGS. 1 and 2.

In FIG. 2, entrainment is done during tachycardia from CS 3-4. The PPI at CS 3-4 is >TCL, indicating CS 3-4 is outside the circuit. EGMs with opposite activation sequence orientation relative to CS 3-4 during entrainments versus native tachycardia are antidromically activated (§). Orthodromically activated sites (those with the same activation sequence relative to CS D during entrainment versus tachycardia are activated orthodromically (§).

Each recording site is analyzed in turn for antidromic activation.

There are two methods of analyzing antidromically activated EGMs.

Method A

Method A relies on measuring the dPPIs of antidromically activated sites.

Once antidromically activated sites are identified, the dPPI is calculated by measuring the interval between the last entrained EGM and the first return EGM at that site.

The observed behavior of dPPIs during entrainment of tachycardias varies depending on whether the tachycardia is focal or re-entrant.

When the tachycardia is focal, the dPPI of antidromically-activated areas approach the TCL as the as that area approaches the point of origin of the tachycardia.

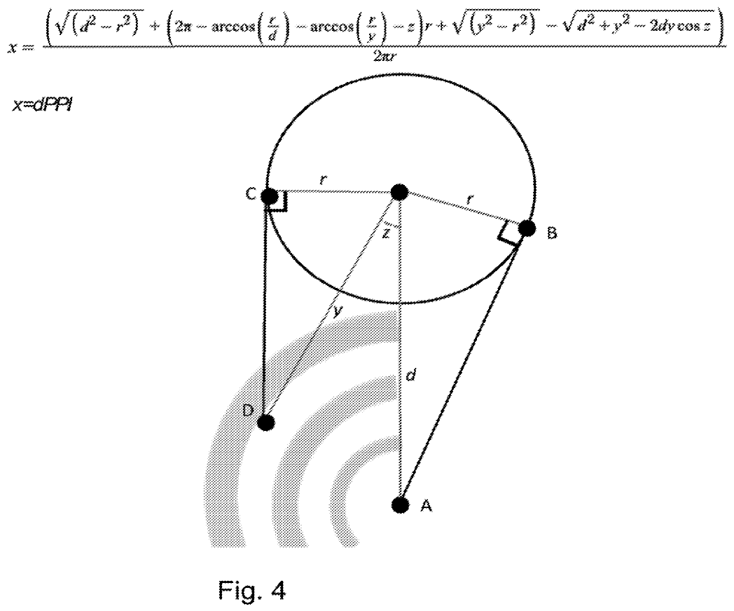

For reentrant tachycardias, the relationship between the pacing location, the recording location, and the radius of the tachycardia circuit can be mathematically approximated by considering the schematic in FIG. 4. In FIG. 4, the formula shows the relationship between the position of the pacing electrode (A), the distance of the pacing electrode to the circuit (d), the radius of the circuit (r), the distance between the recording electrode and the center of the circuit (y), and the derived post-pacing interval (dPPI). The concentric grey semicircles represent the zone of antidromic activation during entrainment.

The formula depicted in FIG. 4 allows for the calculation of the distance (y) of the recording electrode (D) to the center of the circuit, when the remainder of the variables are either measured or assumed.

The information gathered for either focal or reentrant tachycardias can be plotted on an electro-anatomical model to localize tachycardia origins in three dimensions.

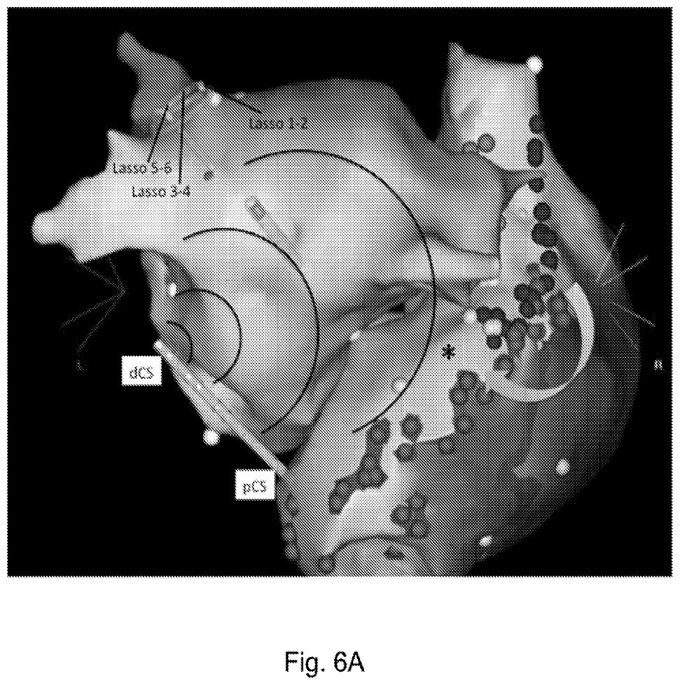

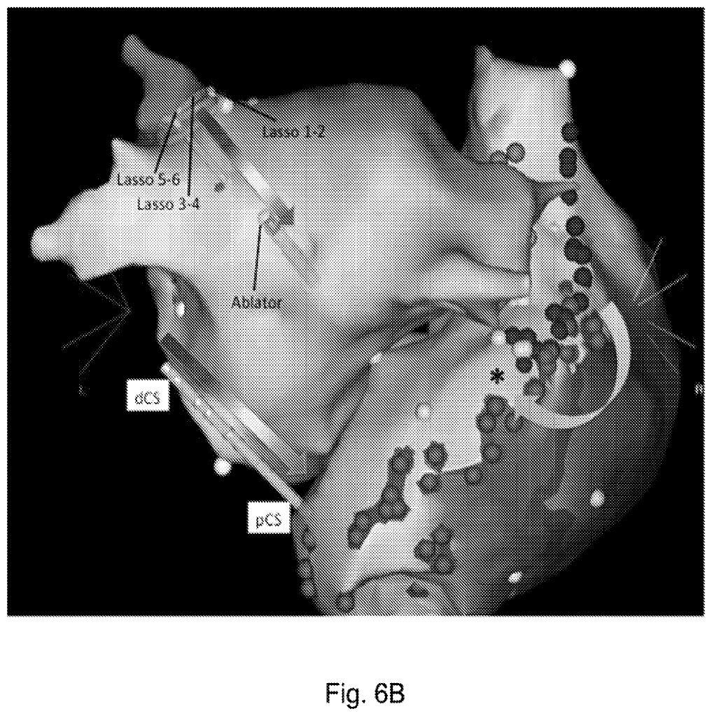

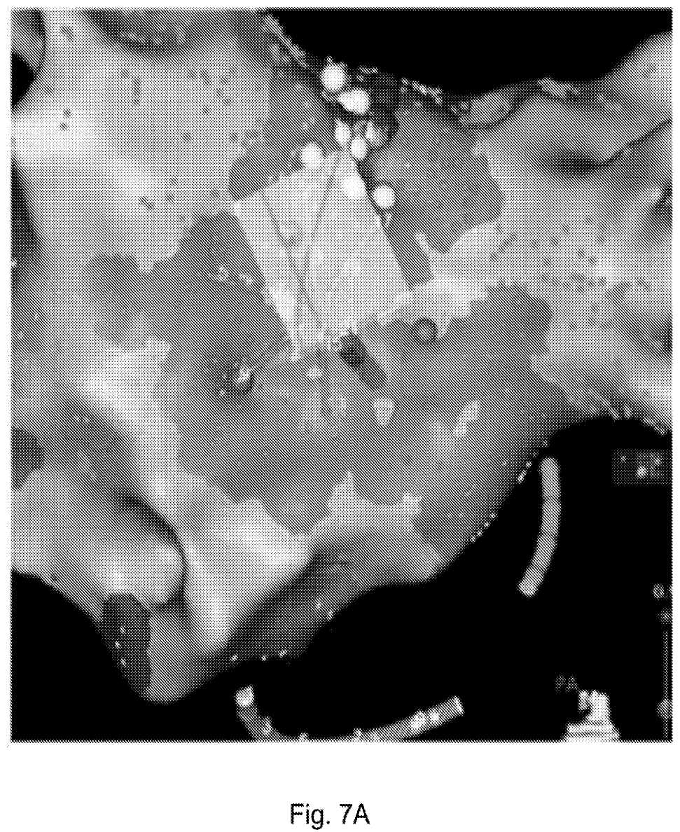

In FIGS. 6A and 6B, Method A has been applied. Entrainment has been performed from the dCS bipole. Antidromic activation recorded at the ablation catheter and the remaining CS bipoles. The dPPIs have been applied, pointing to the origin of tachycardia.

In FIG. 6A, the arrhythmia studied in FIG. 2 was studied with conventional techniques and determined to be rotating around an area of scar in the right atrium. The electroanatomic map of this arrhythmia is shown. The direction of tachycardia activation is shown with the curved arrow. The paced wave front (from dCS) during entrainment is depicted with concentric circles. The intersection between the two is shown (*). dPPIs can be calculated from recording bipoles.

FIG. 6B shows the information obtained in FIGS. 2 displayed graphically on the electroanatomic model, revealing the directions and relative distances of the recording areas to the circuit.

Method B

Method B replies on measuring relative activation of two bipoles as well as the distance between those bipoles. Distance can be directly measured, when it is displayed on an electroanatomical mapping system, or estimated by using antidromic activation during entrainment, or sinus rhythm.

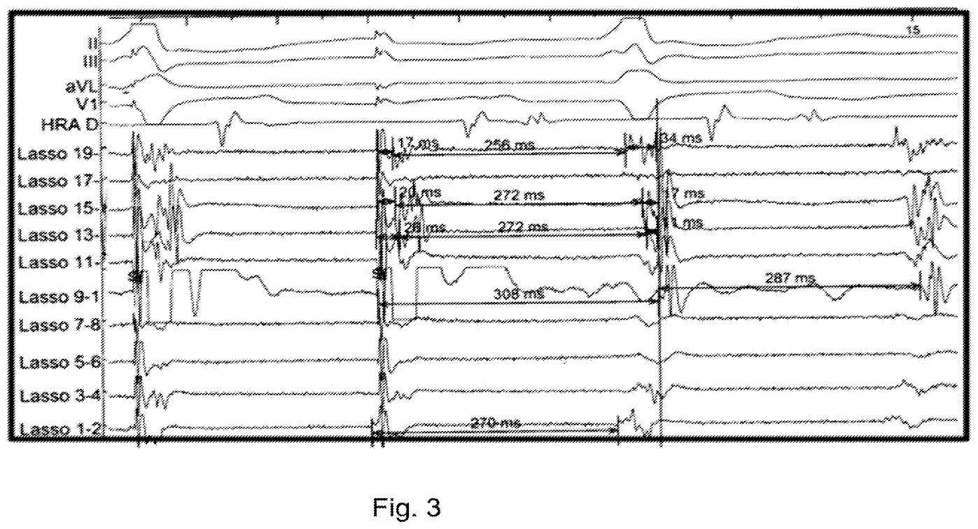

As shown in FIG. 3, in tachycardia, the absolute difference in activation timing between the EGMs of the recording electrode and the electrode used for entrainment is recorded.

Next, the time required for the paced impulse (originating at Lasso 9,10 in FIG. 3) to travel to a recorded EGM is noted. In tachycardia, the time elapsed between the same two bipoles (now activated with opposite orientations) is again recorded. For example, the activation time during pacing between Lassos 9,10 and 13,14 is 26 ms. During tachycardia, it is 11 ms. This can be repeated for all antidromically activated bipoles present. Assuming a constant conduction velocity, timing can be used as a surrogate for distance.

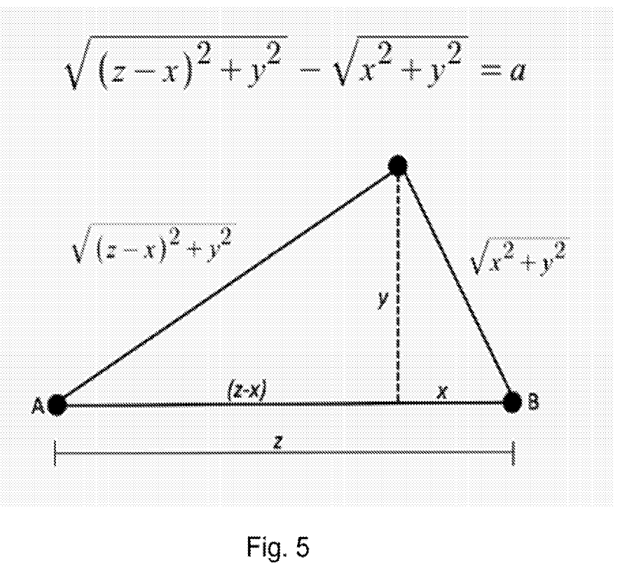

This information is applied as shown in FIG. 5. The time required for an impulse to travel linearly from bipoles B to A is z. The difference in activation time between B and A during tachycardia is a. The formula depicted illustrated the relationship between “z”, “a”, and the origin of the impulse (x, y). The output of this formula generates a tracing that plots a locus of points along which the wave front origin lies.

When this process is repeated for additional bipole pairs, the intersection of the two lines generated by applying the formula will localize the origin of the tachycardia.

Applying this information to a three dimensional electroanatomic model has the potential to rapidly identify the origins of tachycardia.

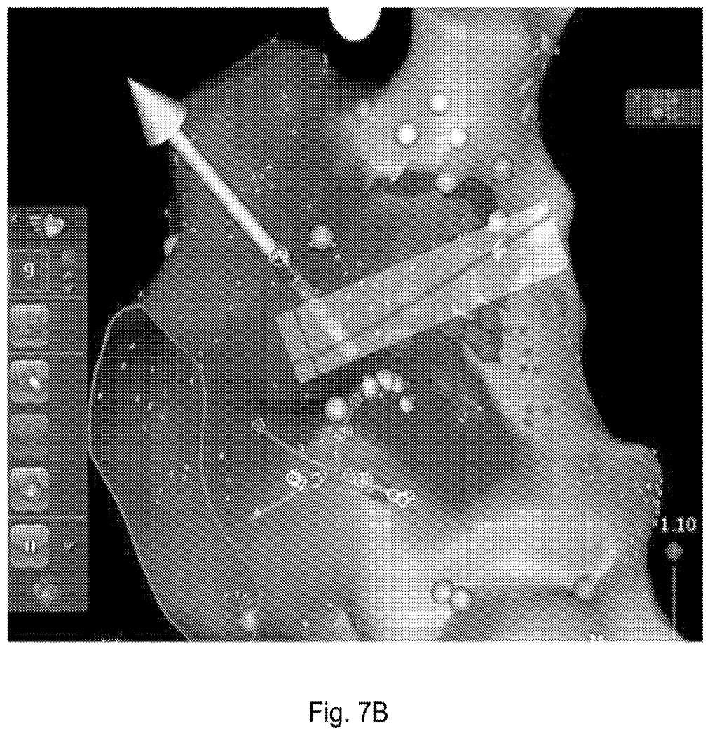

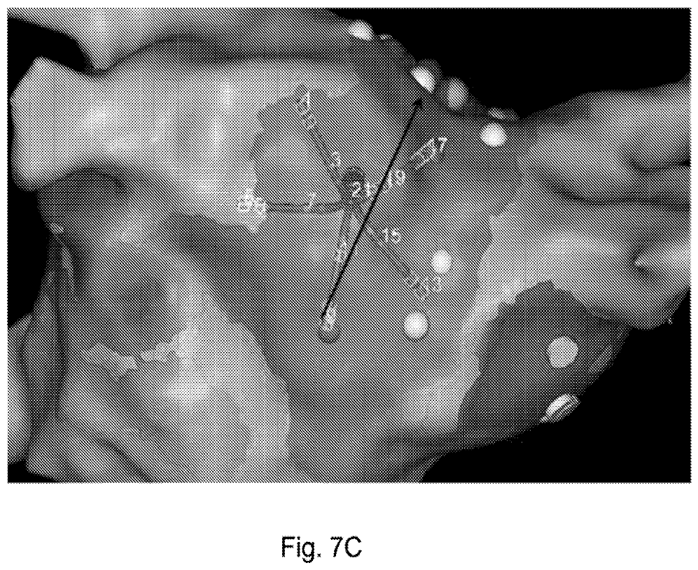

In FIGS. 7A-C, Method B has been applied to three different tachycardias as described.

FIG. 7A shows entrainment being performed from pole 11-12. Using information from recording site 13-14, Method B is utilized to generate a tracing. The origin (exit site) of the tachycardia is predicted to be at some point along the tracing. This tachycardia was successfully ablated at the red dots, directly along the path of the tracing.

FIG. 7B shows entrainment performed from the ablation catheter, and the recording bipole is pentarray 13-14. The output of Method B accurately predicts the origin of the wave front.

In FIG. 7C, the pacing electrode is 9-10, and the recording electrode is 19-20. The output accurately predicts the origin if the tachycardia, which was successfully ablated near the roof of the LA.

The methods have described a way for realizing the value “z”, as shown in FIG. 5. This represents the distance from B to A, measured as the time it takes for an impulse that originates at B to travel to A. As the conduction velocity in tissue is a known constant, the time it takes for the signal to propagate from B to A can be used as a surrogate for a measurement of the distance between B and A. This value can be measured by performing the entrainment maneuver and antidromically activating the tissue between A and B, as described above. However, it should be realized that a measurement of “z” can be taken by antidromically activating the tissue between A and B in the background of any rhythm (not just the tachycardia being studied).

In a preferred embodiment of the invention, the methods discussed can be joined with commercially-available software running on a computer system in communication with the multiple electrodes. The software preferably is capable of providing a three-dimensional visualization of the heart and an accurate measurement of the distance between the pairs of electrodes constituting each bipole.

Using the commercially-available software, the distance from A to B can be measured directly. This eliminates the need to use antidromic activation to deduce how long it takes for an electrical impulse to conduct from A to B. As a result, “z” is measure directly as distance, instead of implying the distance from the time it takes a signal to propagate from B to A.

The value “a” is directly measured as the difference in activation timing between A and B during tachycardia, using the known conduction velocity (which can be a measured or assumed value) to convert that time to a distance. The formula in FIG. 5 is then applied using “a” and “z” as distances.

The output of the formula after inputting “a” and “z” is an equation that states y in terms of x, or in other words, a curve that can be plotted on the three-dimensional model of the heart produced by the software. Applying the formula for subsequent pairs of A and B points yields further curves, and the intersection of the curves indicates the origin of the tachycardia waveform.

I claim:

1. A method of localizing wave fronts in a human heart, comprising:
a. inserting a plurality of electrodes in to said heart;
b. obtaining a measurement of the distance between the electrodes in a pair of said electrodes;
c. measuring the difference in time in the activation of each electrode in said pair of electrodes as said wave front propagates through said heart, and implying a distance from said difference in time;
d. creating a plot of possible locations of the origin of said wave front, based on said distance between said electrodes and said implied distance; and
e. repeating steps (b)-(d) for one or more additional pairs of electrodes, with the origin of said wave front being approximated by the intersection of two or more plots created in step (d).

2. The method of claim 1 wherein said plots are created on a three-dimensional visual model of said heart, created by software.

3. The method of claim 2 wherein said software can provide the measurement of the distance between electrodes in each pair of electrodes.

4. The method of claim 1 wherein said implied distance is based on the known speed of propagation of a wave front through said heart.

5. The method of claim 1 wherein said measurement of the distance between the electrodes in each pair of said electrodes is obtained by direct measuring of said distance.

6. The method of claim 1 wherein said plot of possible locations of the origin of said wave front is created using the formula:
a=\sqrt{(z−X)²+Y²}−\sqrt{X²+Y²})
wherein:
z is the distance between the electrodes in said pair of electrodes;
a is said implied distance based on the difference in time in activation of each electrode in said pair of electrodes; and
X and Y are coordinates of possible locations of said origin of said wave front.

7. The method of claim 1 further comprising the steps of:
a. defining one of said plurality of electrodes as a pacing electrode and the others as recording electrodes;
b. applying a pacing signal to aid pacing electrode until entrainment occurs; and
c. identifying antidromatically activated recording sites.

8. The method of claim 7 further comprising the steps of:
a. obtaining the derived post-pacing interval; and
b. identify a tachycardia wave front by observing a relationship between the derived post-pacing interval of said antidromatically activated areas and the proximity of said antidromatically activated area to the point of origin of the tachycardia.

9. The method of claim 8 wherein said derived post-pacing interval of antidromatically activated areas approaches the tachycardia cycle length as that area approaches said point of origin.

10. The method of claim 8 wherein said derived post-pacing interval is obtained by measuring the time difference between the last entrained electrogram and the first returned electrogram.

11. The method of claim 1 wherein said plots are made on a three dimensional electroanatomical model of said heart to localize the origin of the tachycardia in three dimensions.

12. A method for localizing the origin of a tachycardia in a human heart, comprising:
a. inserting a plurality of electrodes into said heart;
b. applying a pacing signal to one of said electrodes until entrainment occurs;
c. identifying which of said non-pacing electrodes are an antidromatically activated;
d. determining, the time it takes for an impulse to travel between two non-pacing electrodes during linear activation;
e. determining the difference in activation time between said two non-pacing recording electrodes;
f. repeating steps (d) and (e) for multiple pairs of non-pacing electrodes; and
g. mathematically approximating the origin of the tachycardia based on the results of steps (d) and (e) for all pairs of non-pacing electrodes.

13. A system for localizing wave fronts in a human heart, comprising:
a. a plurality of electrodes inserted into said heart;
b. a computer, in communication with each of said electrodes; and
c. software, running on said computer, said software being capable of performing the functions of:
reading and plotting waveforms received at each of said electrodes;
creating and displaying a three-dimensional model of said heart;
providing a measurement of distance between each of said electrodes. measuring the difference in time in the activation of each electrode in a pair of electrodes as said wave front propagates through said heart, and implying a distance from said difference in time;
creating a plot of possible locations of the origin of said wave front on said three-dimensional model of said heart, based on said distance between said electrodes and said implied distance; and
repeating the process for one or more additional pairs of electrodes, with the origin of said wave front being approximated by the intersection of two or more of said created plots.

14. The system of claim 1 wherein said plot of possible locations of the origin of said wave front is created using the formula:
a=\sqrt{(z−X)²+Y²}−\sqrt{X²+Y²}
wherein:
z is the distance between the electrodes in said pair of electrodes;
a is said implied distance based on the difference in time in activation of each electrode in said pair of electrodes; and
X and Y are coordinates of possible locations of said origin of said wave front.
